(19) United States
(12) Patent Application Publication
(10) Pub. No.: US 2023/0001180 A1
(43) Pub. Date: Jan. 5, 2023

(54) SYSTEMS AND METHODS OF DERIVING PRESSURES EXTERNAL TO AN INTRACARDIAC BLOOD PUMP USING INTERNAL PRESSURE SENSORS

(71) Applicant: ABIOMED, Inc., Danvers, MA (US)

(72) Inventors: Stefan Boensch, Aachen (DE); Cem Tozeren, Danvers, MA (US); Ajay Iyer, Danvers, MA (US)

(73) Assignee: ABIOMED, Inc., Danvers, MA (US)

(21) Appl. No.: 17/848,619

(22) Filed: Jun. 24, 2022

Related U.S. Application Data
(60) Provisional application No. 63/216,883, filed on Jun. 30, 2021.

(51) Int. Cl. A61M 60/237; A61M 60/148; A61M 60/178

(52) U.S. Cl. CPC A61M 60/237; A61M 60/148; A61M 60/178

(57) ABSTRACT

Systems and methods for deriving pressures outside of a blood inlets and blood outlets of an intracardiac blood pump assembly, and pressure differentials therebetween. Pressures outside of a blood inlet may be derived based on one or more readings from a pressure sensor placed within a blood inlet, one or more readings from a differential pressure sensor configured to measure pressure differential across a wall of the pump housing or cannula, and speed of the pump motor. Pressure differentials between a blood inlet and blood outlet may be derived based on one or more readings from the differential pressure sensor and speed of the pump motor. Pressures outside of a blood outlet may be derived based on a derived pressure outside of a blood inlet and a derived pressure differential between the blood inlet and the blood outlet.

### CROSS-REFERENCE TO RELATED APPLICATIONS

The present application claims priority to U.S. Provisional Application No. 63/216,883 filed Jun. 30, 2021, the disclosure of which is hereby incorporated by reference herein in its entirety.

### BACKGROUND

Intracardiac blood pump assemblies can be introduced into the heart either surgically or percutaneously and used to deliver blood from one location in the heart or circulatory system to another location in the heart or circulatory system. For example, when deployed in the left heart, an intracardiac blood pump can pump blood from the left ventricle of the heart into the aorta. Likewise, when deployed in the right heart, an intracardiac blood pump can pump blood from the inferior vena cava into the pulmonary artery. Intracardiac pumps can be powered by a motor located outside of the patient's body via an elongate drive shaft (or drive cable) or by an onboard motor located inside the patient's body. Some intracardiac blood pump systems can operate in parallel with the native heart to supplement cardiac output and partially or fully unload components of the heart. Examples of such systems include the IMPELLA® family of devices (Abiomed, Inc., Danvers Mass.).

### BRIEF SUMMARY

The present technology relates to systems and methods for deriving pressures outside of a blood inlet and/or outside of a blood outlet of an intracardiac blood pump assembly, or a pressure differential therebetween. In that regard, the present technology enables pressure outside of the blood inlet to be derived based on one or more readings from a pressure sensor placed within the blood inlet, one or more readings from a differential pressure sensor configured to measure pressure differential across a wall of the pump housing or cannula, and the speed of the pump motor. Likewise, the present technology enables a pressure differential between the blood inlet and blood outlet to be derived based on one or more readings from the differential pressure sensor, and the speed of the pump motor. Further, the pressure outside of the blood outlet may be derived based on the derived pressure outside of the blood inlet and the derived pressure differential between the blood inlet and blood outlet. These derived pressures may be used for a variety of purposes, such as confirming placement of the intracardiac blood pump assembly at a desired position within the patient's heart, and monitoring heart function. Moreover, the systems and methods described herein provide the advantage of allowing inlet and outlet pressures to be derived from pressure sensors placed within the pump housing, where the sensors can also be used to monitor changes in suction which may indicate kinking of the cannula and/or the presence of an obstruction within the pump such as a blood clot.

In one aspect, the disclosure describes an intracardiac blood pump system, comprising an intracardiac blood pump assembly and a controller. The intracardiac blood pump assembly comprises: a motor; a blood inlet; a blood outlet; a cannula positioned between the blood inlet and the blood outlet; a pump housing in fluid communication with the cannula; an impeller positioned within the pump housing and configured to be driven rotationally by the motor; a first pressure sensor configured to measure a pressure differential across a wall of the pump housing or the cannula; and a second pressure sensor positioned within the blood inlet or the pump housing and configured to measure a pressure within the blood inlet or pump housing. The controller comprises a memory, and one or more processors coupled to the memory and configured to: determine a speed of the motor, an output of the first pressure sensor, and an output of the second pressure sensor; determine a first offset value based on the determined speed of the motor, the determined output of the first pressure sensor, and first data, wherein the first data correlates, for each given motor speed of a plurality of motor speeds, a first plurality of differential pressure values to a first plurality of offset values; and determine a first pressure value based on the determined output of the second pressure sensor and the first offset value, wherein the first pressure value represents an estimate of a pressure outside the blood inlet. In some aspects, the one or more processors are further configured to: determine a second offset value based on the determined speed of the motor, the determined output of the first pressure sensor, and second data, wherein the second data is different than the first data and correlates, for each given motor speed of a plurality of motor speeds, a second plurality of differential pressure values to a second plurality of offset values; and determine a second pressure value based on the determined output of the first pressure sensor and the second offset value, wherein the second pressure value represents an estimate of a difference between a pressure outside the blood inlet and a pressure outside the blood outlet. In some aspects, the one or more processors are further configured to determine a third pressure value based on the first pressure value and the second pressure value, wherein the third pressure value represents an estimate of the pressure outside the blood outlet. In some aspects, the intracardiac blood pump assembly is configured for use in a right heart of a patient. In some aspects, the intracardiac blood pump assembly is configured for use in a left heart of a patient. In some aspects, the intracardiac blood pump assembly further comprises: an elongate catheter; and a motor housing positioned between a distal end of the elongate catheter and a proximal end of the blood inlet, wherein the motor housing is configured to house the motor and to be inserted into a patient. In some aspects, the intracardiac blood pump assembly further comprises an elongate catheter, and the motor is configured to drive the impeller via a drive shaft extending through the elongate catheter while the motor remains outside of a patient.

In another aspect, the disclosure describes a method for operating an intracardiac blood pump system, comprising: (a) inserting a portion of an intracardiac blood pump assembly into a patient's heart, the intracardiac blood pump assembly comprising: a motor; a blood inlet; a blood outlet; a cannula positioned between the blood inlet and the blood outlet; a pump housing in fluid communication with the cannula; an impeller positioned within the pump housing and configured to be driven rotationally by the motor; a first pressure sensor configured to measure a pressure differential across a wall of the pump housing or the cannula; and a second pressure sensor positioned within the blood inlet or the pump housing and configured to measure a pressure within the blood inlet or pump housing; (b) operating the intracardiac blood pump assembly to pump blood from the blood inlet to the blood outlet; (c) determining, by one or more processors of a controller, a speed of the motor, an output of the first pressure sensor, and an output of the second pressure sensor; (d) determining, by the one or more processors, a first offset value based on the determined speed of the motor, the determined output of the first pressure sensor, and first data, wherein the first data correlates, for each given motor speed of a plurality of motor speeds, a first plurality of differential pressure values to a first plurality of offset values; and (e) determining, by the one or more processors, a first pressure value based on the determined output of the second pressure sensor and the first offset value, wherein the first pressure value represents an estimate of a pressure outside the blood inlet. In some aspects, inserting the portion of an intracardiac blood pump assembly into a patient's heart comprises positioning the blood inlet within the patient's inferior vena cava and the blood outlet within the patient's pulmonary artery, and the first pressure value represents an estimate of the patient's central venous pressure. In some aspects, inserting the portion of an intracardiac blood pump assembly into a patient's heart comprises positioning the blood inlet within the patient's left ventricle and the blood outlet within the patient's ascending aorta, and the first pressure value represents an estimate of the patient's left ventricular pressure. In some aspects, the method further comprises: (f) determining, by the one or more processors, a second offset value based on the determined speed of the motor, the determined output of the first pressure sensor, and second data, wherein the second data is different than the first data and correlates, for each given motor speed of a plurality of motor speeds, a second plurality of differential pressure values to a second plurality of offset values; and (g) determining, by the one or more processors, a second pressure value based on the determined output of the first pressure sensor and the second offset value, wherein the second pressure value represents an estimate of a difference between a pressure outside the blood inlet and a pressure outside the blood outlet. In some aspects, the method further comprises: (h) determining, by the one or more processors, a third pressure value based on the first pressure value and the second pressure value, wherein the third pressure value represents an estimate of the pressure outside the blood outlet. In some aspects, inserting the portion of an intracardiac blood pump assembly into a patient's heart comprises positioning the blood inlet within the patient's inferior vena cava and the blood outlet within the patient's pulmonary artery, and the third pressure value represents an estimate of the patient's pulmonary artery pressure. In some aspects, inserting the portion of an intracardiac blood pump assembly into a patient's heart comprises positioning the blood inlet within the patient's left ventricle and the blood outlet within the patient's ascending aorta, and the first pressure value represents an estimate of the patient's central aortic pressure. In some aspects, the intracardiac blood pump assembly further comprises an elongate catheter and a motor housing positioned between a distal end of the elongate catheter and a proximal end of the blood inlet, and configured to house the motor; and inserting a portion of an intracardiac blood pump assembly into a patient's heart comprises inserting the motor housing into the patient. In some aspects, the intracardiac blood pump assembly further comprises an elongate catheter, and operating the intracardiac blood pump assembly to pump blood from the blood inlet to the blood outlet comprises driving the impeller via a drive shaft extending through the elongate catheter while the motor remains outside of a patient.

### BRIEF DESCRIPTION OF DRAWINGS

FIG. 1 depicts an exemplary intracardiac blood pump assembly configured for left heart support, in accordance with aspects of the disclosure.

FIG. 2 depicts an exemplary intracardiac blood pump assembly configured for right heart support, in accordance with aspects of the disclosure.

FIG. 3 is a functional block diagram of an exemplary system, in accordance with aspects of the disclosure.

FIG. 4 depicts a cross-sectional view of a portion of the intracardiac blood pump assembly of FIG. 2 with an exemplary placement for the differential pressure sensor and inlet pressure sensor of FIG. 3, in accordance with aspects of the disclosure.

FIG. 5 is a flow diagram of an exemplary method for determining pressure at a point outside of a blood inflow cage of an intracardiac blood pump assembly, in accordance with aspects of the disclosure.

FIG. 6 is a chart showing a set of exemplary inlet pressure offset curves, in accordance with aspects of the disclosure.

FIG. 7 is a flow diagram of an exemplary method for determining an external pressure differential between a point outside of a blood inflow cage and a point outside of a blood outflow cage of an intracardiac blood pump assembly, in accordance with aspects of the disclosure.

FIGS. 8A and 8B are charts showing a set of exemplary external differential pressure offset curves, in accordance with aspects of the disclosure.

FIG. 9 is a flow diagram of an exemplary method for determining pressure at a point outside of a blood outflow cage of an intracardiac blood pump assembly, in accordance with aspects of the disclosure.

### DETAILED DESCRIPTION

Embodiments of the present disclosure are described in detail with reference to the figures wherein like reference numerals identify similar or identical elements. It is to be understood that the disclosed embodiments are merely examples of the disclosure, which may be embodied in various forms. Well known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

To provide an overall understanding of the systems, methods, and devices described herein, certain illustrative examples will be described. Although various examples may describe intracardiac blood pump assemblies, it will be understood that the improvements of the present technology may also be adapted and applied to other types of medical devices such as electrophysiology study and catheter ablation devices, angioplasty and stenting devices, angiographic catheters, peripherally inserted central catheters, central venous catheters, midline catheters, peripheral catheters, inferior vena cava filters, abdominal aortic aneurysm therapy devices, thrombectomy devices, TAVR delivery systems, cardiac therapy and cardiac assist devices, including balloon pumps, cardiac assist devices implanted using a surgical incision, and any other venous or arterial based introduced catheters and devices.

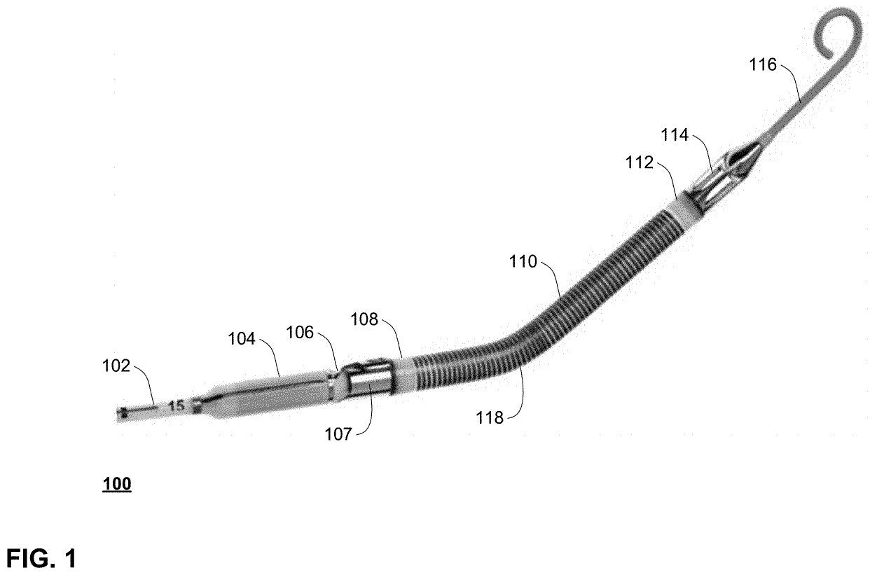

FIG. 1 depicts an exemplary intracardiac blood pump assembly 100 adapted for left heart support, in accordance with aspects of the disclosure. In that regard, the intracardiac blood pump assembly 100 includes an elongate catheter 102, a motor 104, a cannula 110, a blood inflow cage 114 arranged at or near the distal end 112 of the cannula 110, a blood outflow cage 106 arranged at or near the proximal end 108 of the cannula 110, and an optional atraumatic extension 116 arranged at the distal end of the blood inflow cage 114.

Motor 104 is configured to rotatably drive an impeller (not shown), thereby generating suction sufficient to draw blood into cannula 110 through the blood inflow cage 114, and to expel the blood out of cannula 110 through the blood outflow cage 106. In that regard, the impeller may be positioned distal of the blood outflow cage 106, for example, within the proximal end 108 of the cannula 110 or within a pump housing 107 coupled to the proximal end 108 of the cannula 110. In some aspects of the technology, rather than the impeller being driven by an in-dwelling motor 104, the impeller may instead be coupled to an elongate drive shaft (or drive cable) which is driven by a motor located external to the patient.

Catheter 102 may house electrical lines coupling the motor 104 to one or more electrical controllers or other sensors. Alternatively, where the impeller is driven by an external motor, an elongate drive shaft may pass through catheter 102. Catheter 102 may also serve as a conduit for wires connecting the pressure sensors described further below to one or more controllers, power sources, etc. located outside the patient's body. Catheter 102 may also include a purge fluid conduit, a lumen configured to receive a guidewire, etc.

The blood inflow cage 114 includes one or more apertures or openings configured to allow blood to be drawn into cannula 110 when the motor 104 is operating. Likewise, blood outflow cage 106 includes one or more apertures or openings configured to allow blood to flow from the cannula 110 out of the intracardiac blood pump assembly 100. Blood inflow cage 114 and outflow cage 106 may be composed of any suitable bio-compatible material(s). For example, blood inflow cage 114 and/or blood outflow cage 106 may be formed out of bio-compatible metals such as stainless steel, titanium, or biocompatible polymers such as polyurethane. In addition, the surfaces of blood inflow cage 114 and/or blood outflow cage 106 may be treated in various ways, including, but not limited to etching, texturing, or coating or plating with another material. For example, the surfaces of blood inflow cage 114 and/or blood outflow cage 106 may be laser textured.

Cannula 110 may include a flexible hose portion. For example, cannula 110 may be composed, at least in part, of a polyurethane material. In addition, cannula 110 may include a shape-memory material. For example, cannula 110 may comprise a combination of a polyurethane material and one or more strands or coils of a shape-memory material such as Nitinol. Cannula 110 may be formed such that it includes one or more bends or curves in its relaxed state, or it may be configured to be straight in its relaxed state. In that regard, in the exemplary arrangement shown in FIG. 1, the cannula 110 has a single pre-formed anatomical bend 118 based on the portion of the left heart in which it is intended to operate. Despite this bend 118, the cannula 110 may nevertheless also be flexible, and may thus be capable of straightening (e.g., during insertion over a guidewire), or bending further (e.g., in a patient whose anatomy has tighter dimensions). Further in that regard, cannula 110 may include a shape-memory material configured to allow the cannula 110 to be a different shape (e.g., straight or mostly straight) at room temperatures, and to form bend 118 once the shape-memory material is exposed to the heat of a patient's body.

Atraumatic extension 116 assists with stabilizing and positioning the intracardiac blood pump assembly 100 in the correct position in the patient's heart. Atraumatic extension 116 may be solid or tubular. If tubular, atraumatic extension 116 may be configured to allow a guidewire to be passed through it to further assist in the positioning of the intracardiac blood pump assembly 100. Atraumatic extension 116 may be any suitable size. For example, atraumatic extension 116 may have an outer diameter in the range of 4-8 Fr. Atraumatic extension 116 may be composed, at least in part, of a flexible material, and may be any suitable shape or configuration such as a straight configuration, a partially curved configuration, a pigtail-shaped configuration as shown in the example of FIG. 1, etc. Atraumatic extension 116 may also have sections with different stiffnesses. For example, atraumatic extension 116 may include a proximal section that is stiff enough to prevent it from buckling, thereby keeping the blood inflow cage 114 in the desired location, and a distal section that is softer and has a lower stiffness, thereby providing an atraumatic tip for contact with a wall of the patient's heart and to allow for guidewire loading. In such a case, the proximal and distal sections of the atraumatic extension 116 may be composed of different materials, or may be composed of the same material, treated to provide different stiffnesses.

Notwithstanding the foregoing, as mentioned above, atraumatic extension 116 is an optional structure. In that regard, the present technology may also be used with intracardiac blood pump assemblies and other intracardiac devices that include extensions of different types, shapes, materials, and qualities. Likewise, the present technology may be used with intracardiac blood pump assemblies and other intracardiac devices that have no distal extensions of any kind.

Intracardiac blood pump assembly 100 may be inserted percutaneously. For example, when used for left heart support, intracardiac blood pump assembly 100 may be inserted via a catheterization procedure through the femoral artery or axillary artery, into the aorta, across the aortic valve, and into the left ventricle. Once positioned in this way, the intracardiac blood pump assembly 100 delivers blood from the blood inflow cage 114, which sits inside the left ventricle, through cannula 110, to the blood outflow cage 106, which sits inside the ascending aorta. In some aspects of the technology, intracardiac blood pump assembly 100 may be configured such that bend 118 will rest against a predetermined portion of the patient's heart when the intracardiac blood pump assembly 100 is in a desired location. Likewise, the atraumatic extension 116 may be configured such that it rests against a different predetermined portion of the patient's heart when the intracardiac blood pump assembly 100 is in the desired location.

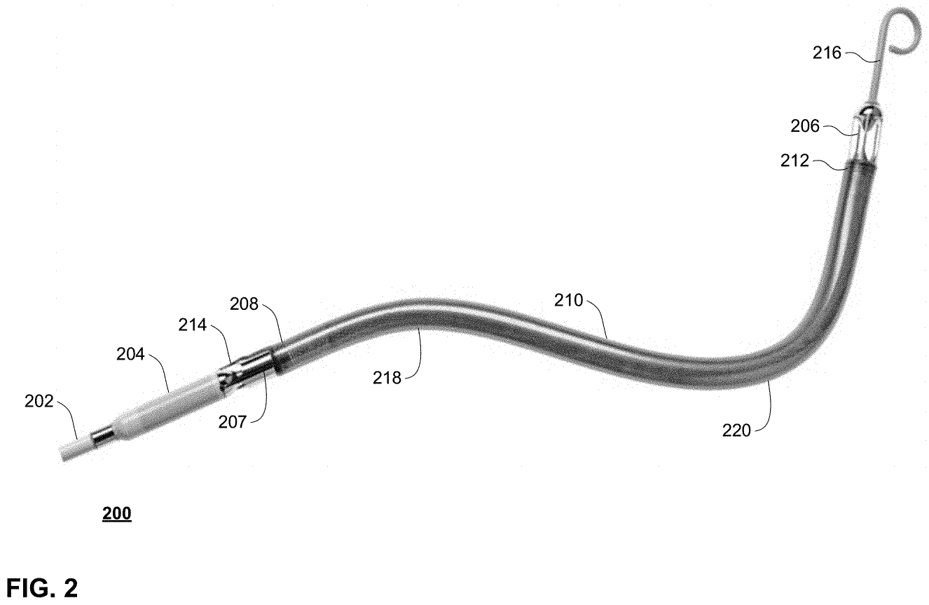

FIG. 2 depicts an exemplary intracardiac blood pump assembly 200 adapted for right heart support, in accordance with aspects of the disclosure. In that regard, the intracardiac blood pump assembly 200 includes an elongate catheter 202, a motor 204, a cannula 210, a blood inflow cage 214 arranged at or near the proximal end 208 of the cannula 210, a blood outflow cage 206 arranged at or near the distal end 212 of the cannula 210, and an optional atraumatic extension 216 arranged at the distal end of the blood outflow cage 206.

As with the exemplary assembly of FIG. 1, motor 204 is configured to rotatably drive an impeller (not shown), thereby generating suction sufficient to draw blood into cannula 210 through the blood inflow cage 214, and to expel the blood out of cannula 210 through the blood outflow cage 206. In that regard, the impeller may be positioned distal of the blood inflow cage 214, for example, within the proximal end 208 of the cannula 210 or within a pump housing 207 coupled to the proximal end 208 of the cannula 210. Here as well, in some aspects of the technology, rather than the impeller being driven by an in-dwelling motor 204, the impeller may instead be coupled to an elongate drive shaft (or drive cable) which is driven by a motor located external to the patient.

The cannula 210 of FIG. 2 serves the same purpose, and may have the same properties and features described above with respect to cannula 110 of FIG. 1. However, in the exemplary arrangement shown in FIG. 2, the cannula 210 has two pre-formed anatomical bends 218 and 220 based on the portion of the right heart in which it is intended to operate. Here again, despite the existence of bends 218 and 220, the cannula 210 may nevertheless also be flexible, and may thus be capable of straightening (e.g., during insertion over a guidewire), or bending further (e.g., in a patient whose anatomy has tighter dimensions). Further in that regard, cannula 210 may include a shape-memory material configured to allow the cannula 210 to be a different shape (e.g., straight or mostly straight) at room temperatures, and to form bends 218 and/or 220 once the shape-memory material is exposed to the heat of a patient's body.

The catheter 202 and atraumatic extension 216 of FIG. 2 serve the same purpose and may have the same properties and features described above with respect to catheter 102 and atraumatic extension 116 of FIG. 1. Likewise, other than being located at opposite ends of the cannula from those of FIG. 1, the blood inflow cage 214 and blood outflow cage 206 of FIG. 2 are similar to the blood inflow cage 114 and blood outflow cage 106 of FIG. 1, and thus may have the same properties and features described above.

Like the exemplary assembly of FIG. 1, the intracardiac blood pump assembly 200 of FIG. 2 may also be inserted percutaneously. For example, when used for right heart support, intracardiac blood pump assembly 200 may be inserted via a catheterization procedure through the femoral vein, into the inferior vena cava, through the right atrium, across the tricuspid valve, into the right ventricle, through the pulmonary valve, and into the pulmonary artery. Once positioned in this way, the intracardiac blood pump assembly 200 delivers blood from the blood inflow cage 214, which sits inside the inferior vena cava, through cannula 210, to the blood outflow cage 206, which sits inside the pulmonary artery.

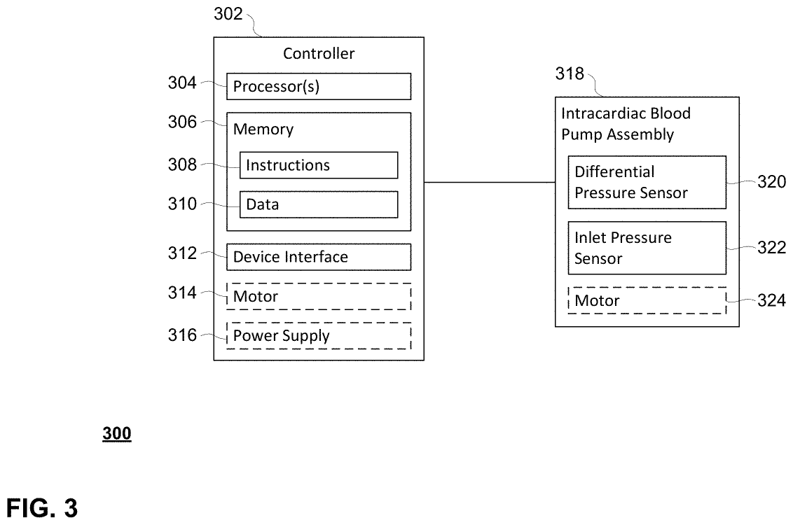

FIG. 3 is a functional block diagram of an exemplary system, in accordance with aspects of the disclosure. In that regard, in the example of FIG. 3, the system 300 comprises an intracardiac blood pump assembly 318 and a controller 302. The intracardiac blood pump assembly 318 may take any form, including those shown in the exemplary blood pump assemblies 100 and 200 of FIG. 1 or 2, respectively. In addition, the intracardiac blood pump assembly 318 of FIG. 3 includes a differential pressure sensor 320 configured to measure pressure differential across a wall of the pump housing (e.g., pump housing 107 or 207) or cannula (e.g., cannula 110 or 210), and an inlet pressure sensor 322 configured to measure pressure within the pump near the blood inlet. For example, inlet pressure sensor 322 may be positioned within the blood inflow cage (e.g., blood inflow cage 114 or 214), or within a portion of the pump housing (e.g., pump housing 107 or 207). As shown in FIG. 3, the intracardiac blood pump assembly 318 may additionally include an optional motor 324 configured to rotatably drive an impeller (e.g., in instances where the motor is configured to be inserted into the patient). Notwithstanding the foregoing, the present technology may also be employed in systems comprising an intracardiac device other than a blood pump assembly.

In the example of FIG. 3, the controller 302 includes or more processors 304 coupled to memory 306 storing instructions 308 and data 310, and an interface 312 with the intracardiac blood pump assembly 318. Controller 302 may additionally include an optional motor 314 (e.g., in instances where the impeller is driven by a motor located external to the patient via an elongate drive shaft) and/or a power supply 316 (e.g., to power an in-dwelling motor 324, any sensors which require power, etc.). The device interface 312 represents the interface between controller 302 and intracardiac blood pump assembly 318, and may include any suitable type of interface. In that regard, device interface 312 may be configured to enable one-way or two-way communication between the controller 302 and the intracardiac blood pump assembly 318, and may further be configured to receive signals from differential pressure sensor 320 and inlet pressure sensor 322. Interface 312 may further be configured to provide power to an in-dwelling motor 324 and/or one or more sensors (e.g., differential pressure sensor 320, inlet pressure sensor 322, etc.).

Controller 302 may take any form. In that regard, controller 302 may comprise a single modular unit, or its components may be distributed between two or more physical units. Controller 302 may further include any other components normally used in connection with a computing device such as a user interface. In that regard, controller 302 may have a user interface that includes one or more user inputs (e.g., buttons, touchscreen, keypad, keyboard, mouse, microphone, etc.); one or more electronic displays (e.g., a monitor having a screen or any other electrical device that is operable to display information, one or more lights, etc.); one or more speakers, chimes or other audio output devices; and/or one or more other output devices such as vibrating, pulsing, or haptic elements.

The one or more processors 304 and memory 306 described herein may be implemented on any type of computing device(s), including customized hardware or any type of general computing device. Memory 306 may be of any non-transitory type capable of storing information accessible by the processor(s) 304, such as a hard-drive, memory card, optical disk, solid-state drive, tape memory, or similar structure.

Instructions 308 may include programming configured to receive and process readings from the differential pressure sensor 320 and inlet pressure sensor 322. In that regard, instructions 308 may include the programming necessary to calculate the offset values described below. Controller 302 may further be configured to store readings from the differential pressure sensor 320 and inlet pressure sensor 322 in memory 306.

Data 310 may include data for calibrating and/or interpreting the signals of the differential pressure sensor 320 and inlet pressure sensor 322, as well as the look-up tables used in the calculation of the offset values, as described below.

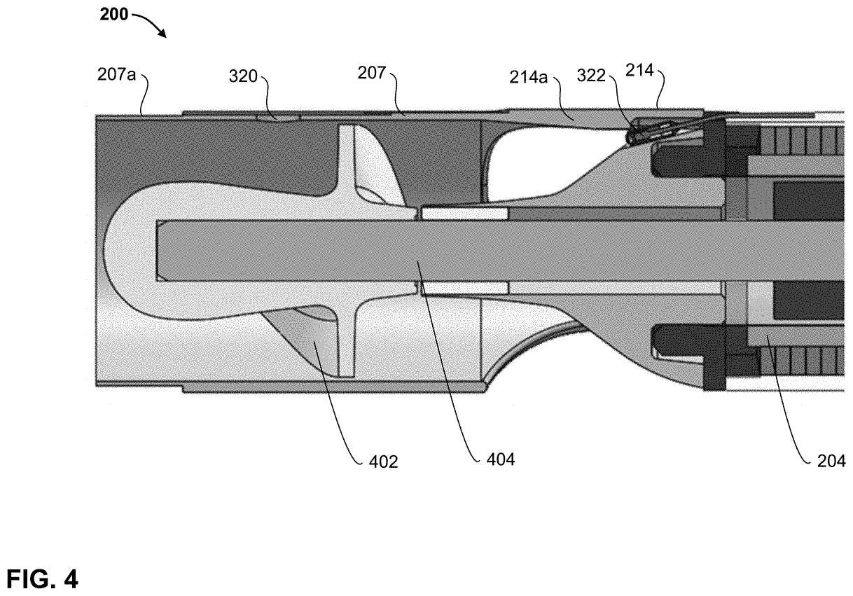

FIG. 4 depicts a cross-sectional view of a portion of the intracardiac blood pump assembly 200 of FIG. 2 with an exemplary placement for the differential pressure sensor 320 and inlet pressure sensor 322 of FIG. 3, in accordance with aspects of the disclosure. In that regard, FIG. 4 shows a cross-section taken along the longitudinal axis of the blood inflow cage 214 and pump housing 207, and depicts an arrangement in which an impeller 402 is configured to be driven rotatably by motor 204 via drive shaft 404. In the example of FIG. 4, the outer diameter of the pump housing 207 is slightly reduced at the distal end 207a, so that it may be inserted into the proximal end 208 of cannula 210 (not shown). In addition, the cross-sectional view of FIG. 4 depicts a strut 214a of the blood inflow cage 214. Although only one such strut is visible in this cross-section, there may be two or more struts 214a.

In the example of FIG. 4, the differential pressure sensor 320 is mounted in a wall of pump housing 207 such that it can measure a pressure differential between the fluid inside and outside of the pump housing 207. In this example, pressure sensor 320 is mounted at or near the midline of the impeller 402. However, differential pressure sensor 320 may also be mounted distal or proximal of impeller 402, e.g., in a different portion of pump housing 207 or in cannula 210.

In the example of FIG. 4, inlet pressure sensor 322 is mounted within the blood inflow cage 214, and is positioned underneath strut 214a. However, inlet pressure sensor 322 may also be positioned in any other suitable portion of the blood inflow cage 214 or pump housing 207. Positioning the inlet pressure sensor 322 inside of the pump in this way provides various advantages. For example, mounting inlet pressure sensor 322 internally may protect it from being bumped or otherwise making contact with portions of the patient's anatomy. In addition, as noted above, this positioning allows the inlet pressure sensor 322 to be used to monitor pressure inside the pump, which can help identify changes in suction that may indicate kinking of the cannula 210 and/or the presence of an obstruction (e.g., blood clot, tissue) within the blood inflow cage 214, pump housing 207, cannula 210, and/or blood outflow cage 206.

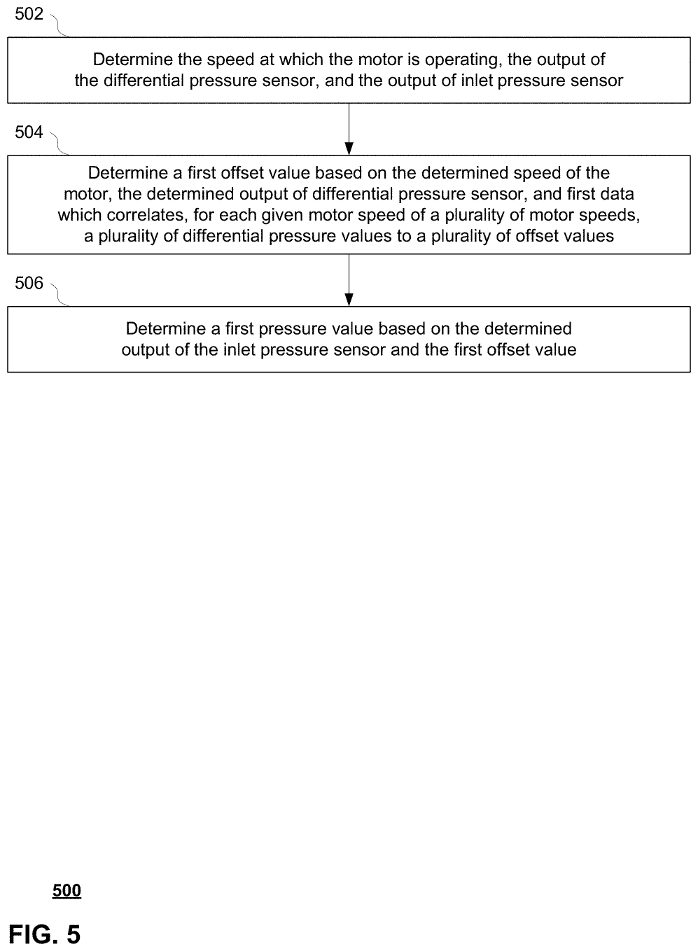

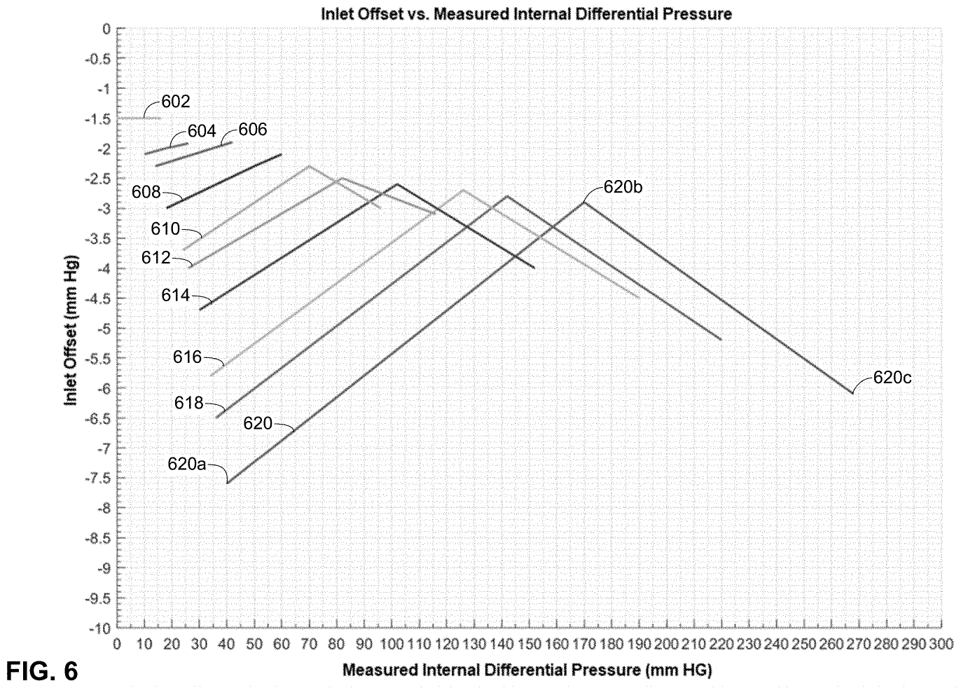

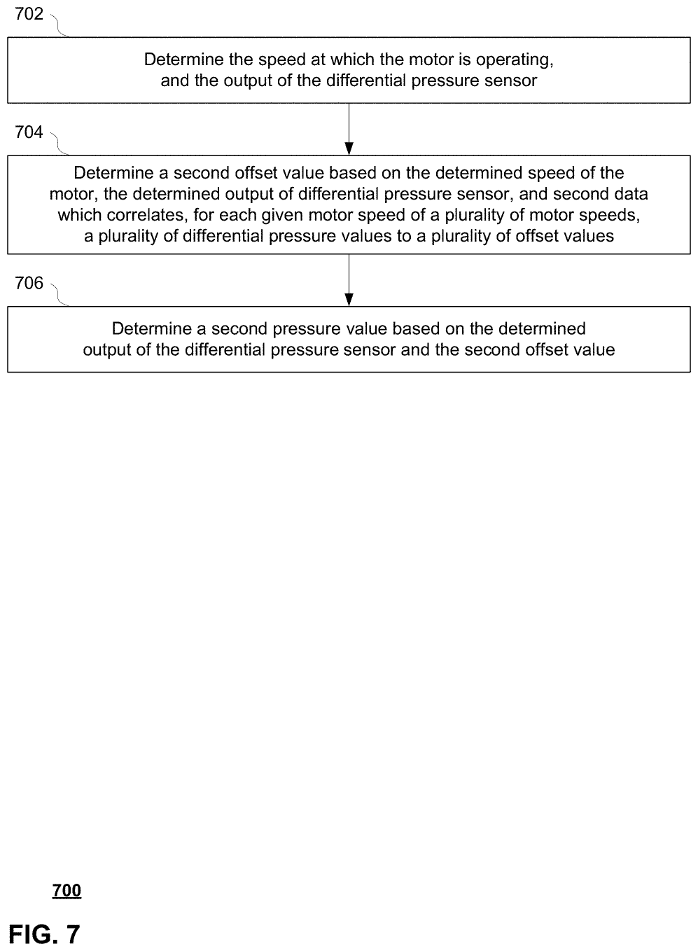

Notwithstanding these advantages, placing the inlet pressure sensor 322 inside the pump can expose it to Venturi effects as blood is pulled into the pump housing 207 by impeller 402, which may cause the pressure readings to be different than they would be if the inlet pressure sensor 322 were instead mounted on an outer surface of blood inflow cage 214, pump housing 207, or the housing of motor 204. As such, in order to use the readings from inlet pressure sensor 322 to derive the pressure of the blood outside of the blood inflow cage 214 (e.g., the central venous pressure (“CVP”) when the intracardiac blood pump is being used to provide right heart support), a first offset value must be calculated based on the pump speed and the differential pressure reading, as described further below with respect to FIGS. 5 and 6. Likewise, in order to use the readings from the differential pressure sensor 320 and the inlet pressure sensor 322 to derive the pressure differential between the pump inlet and outlet, or the pressure of the blood outside of the blood outflow cage 206 (e.g., the pulmonary artery pressure (“PAP”) when the intracardiac blood pump is being used to provide right heart support), a second offset value must be calculated based on the pump speed and the differential pressure reading, as described further below with respect to FIGS. 7-9.

Notably, while it would also be possible to measure CVP and PAP directly using additional pressure sensors mounted on the outside of the pump near the blood inflow cage 214 and the blood outflow cage 206, the present technology advantageously allows for these values to be accurately derived using only the differential pressure sensor 320 and the inlet pressure sensor 322, while also enabling the inlet pressure sensor 322 to be used for directly monitoring suction inside the pump as already described. The present technology thus allows for fewer sensors to be used, resulting in lower costs, fewer potential failure points, and the ability to use the intracardiac blood pump assembly 200 with a larger array of potential controllers, some of which may not have enough inputs to support additional externally mounted pressure sensors.

FIG. 5 is a flow diagram of an exemplary method 500 for determining pressure at a point outside of a blood inflow cage of an intracardiac blood pump assembly, in accordance with aspects of the disclosure. In that regard, in method 500 it is assumed that an intracardiac blood pump assembly (e.g., intracardiac blood pump assembly 200 of FIGS. 2-4) has been inserted into a patient's right heart such that the blood inflow cage (e.g., blood inflow cage 214) is positioned within the patient's inferior vena cava, and the blood outflow cage (e.g., blood outflow cage 216) is positioned within the patient's pulmonary artery, and such that the determined pressure outside of the blood inflow cage will represent the patient's CVP. However, as will be understood, the present technology may likewise be employed in the context of providing left-heart support, in which case the differential pressure sensor (e.g., differential pressure sensor 320) and the inlet pressure sensor (e.g., inlet pressure sensor 322) may be positioned similarly with respect to the blood inflow cage (e.g., blood inflow cage 114), cannula (e.g., cannula 110), and pump housing (e.g., pump housing 107) of an intracardiac blood pump assembly configured for left-heart support (e.g., exemplary intracardiac blood pump assembly 100), and method 500 may be used to instead calculate pressure within the patient's left ventricle (“LVP”).

In step 502, the controller (e.g., controller 302 of FIG. 3) determines the speed at which the motor is operating, the output of the differential pressure sensor, and the output of inlet pressure sensor. In that regard, the controller may determine the speed at which the motor is operating in any suitable way. In some aspects of the technology, motor speed may be determined from the output of a speed sensor configured to monitor the revolutions per minute (or other suitable metric of rotational speed) of the motor, the drive shaft (e.g., drive shaft 404), or the impeller (e.g., impeller 402). In some aspects of the technology, motor speed may be determined from the voltage being applied to the motor and/or the current being drawn by the motor. In some aspects of the technology, the controller may be configured to drive the motor at one of a plurality of preset speeds, in which case the motor speed may be determined based on whatever preset speed is selected.

In step 504, the controller determines a first offset value based on the determined speed of the motor, the determined output of the differential pressure sensor, and first data. The first offset value represents the amount by which the reading of the inlet pressure sensor is expected to differ from the actual pressure outside of the blood inflow cage of the intracardiac blood pump assembly, which may be influenced by various factors such as Venturi effects as blood is drawn into the pump housing (e.g., pump housing 207) by the impeller (e.g., impeller 402), turbulence as blood flows past the struts (e.g., strut 214a) of the blood inflow cage, etc. The first data correlates, for each given motor speed of a plurality of motor speeds, a plurality of differential pressure values to a plurality of offset values. The first data may be represented in any suitable way. Moreover, any suitable manner of estimating, averaging, or interpolating may be used to determine the first offset value based on the determined speed of the motor, the determined output of the differential pressure sensor, and the first data. In that regard, the following examples are intended to be illustrative and nonlimiting.

Thus, in some aspects of the technology, the first data may comprise a lookup table in which each given row corresponds to a given motor speed of a plurality of possible motor speeds, and the columns of each given row represent a plurality of data points correlating a differential pressure value with an offset value. Such lookup table may be represented as a matrix, vector, database, 2D plot, or any other suitable data structure. In such a case, the controller may determine the first offset value by choosing a given row of the lookup table based on the determined speed of the motor (e.g., choosing the row closest to the determined speed of the motor), and interpolating between two data points of the row based on the determined output of the differential pressure sensor. Likewise, the controller may determine the first offset value by choosing a first and second row of the lookup table based on the determined speed of the motor (e.g., choosing the two rows immediately above and below the determined speed of the motor), interpolating between two data points of the first row based on the determined output of the differential pressure sensor to obtain a first estimated offset, interpolating between two data points of the second row based on the determined output of the differential pressure sensor to obtain a second estimated offset, and interpolating between the first estimated offset and the second estimated offset based on the determined speed of the motor. Further, the controller may determine the first offset value by choosing a first and second row of the lookup table based on the determined speed of the motor (e.g., choosing the two rows immediately above and below the determined speed of the motor), generating an interpolated lookup table based on the first and second row of the lookup table and the determined speed of the motor, and interpolating between two data points of the interpolated lookup table based on the determined output of the differential pressure sensor.

Likewise, in some aspects of the technology, the first data may comprise a plurality of lookup tables, each given lookup table representing a different given motor speed of the plurality of motor speeds. Again, each individual lookup table may be represented as a matrix, vector, database, 2D plot, or any other suitable data structure. In such a case, the controller may determine the first offset value by choosing a given lookup table based on the determined speed of the motor (e.g., choosing the lookup table representing the speed closest to the determined speed of the motor), and interpolating between two data points of the given lookup table based on the determined output of the differential pressure sensor. Likewise, the controller may determine the first offset value by choosing a first lookup table and a second lookup table based on the determined speed of the motor (e.g., choosing the lookup tables with speeds immediately above and below the determined speed of the motor), interpolating between two data points of the first lookup table based on the determined output of the differential pressure sensor to obtain a first estimated offset, interpolating between two data points of the second lookup table based on the determined output of the differential pressure sensor to obtain a second estimated offset, and interpolating between the first estimated offset and the second estimated offset based on the determined speed of the motor. Further, the controller may determine the first offset value by choosing a first lookup table and a second lookup table based on the determined speed of the motor (e.g., choosing the lookup tables with speeds immediately above and below the determined speed of the motor), generating an interpolated lookup table based on the first and second lookup tables and the determined speed of the motor, and interpolating between two data points of the interpolated lookup table based on the determined output of the differential pressure sensor.

Further, in some aspects of the technology, the first data may comprise a plurality of functions, each given function of the plurality of functions corresponding to a given motor speed of the plurality of motor speeds, and each given function enabling the calculation of an offset value based on a given differential pressure value. These functions may be generated in any suitable way, such as by performing a regression analysis on a plurality of datapoints. In such a case, the controller may determine the first offset value by choosing a given function based on the determined speed of the motor (e.g., choosing the function representing the speed closest to the determined speed of the motor), and calculating the first offset value using the given function and the determined output of the differential pressure sensor. Likewise, the controller may determine the first offset value by choosing a first function and a second function based on the determined speed of the motor (e.g., choosing the functions with speeds immediately above and below the determined speed of the motor), calculating a first estimated offset using the first function and the determined output of the differential pressure sensor, calculating a second estimated offset using the second function and the determined output of the differential pressure sensor, and interpolating between the first estimated offset and the second estimated offset based on the determined speed of the motor. Further, the controller may determine the first offset value by choosing a first function and a second function based on the determined speed of the motor (e.g., choosing the functions with speeds immediately above and below the determined speed of the motor), generating a third function based on the first and second functions and the determined speed of the motor, and calculating the first offset value using the third function and the determined output of the differential pressure sensor.

In step 506, the controller determines a first pressure value based on the determined output of the inlet pressure sensor and the first offset value. The controller may make this determination in any suitable way. For example, in some aspects of the technology, the controller may determine the first pressure value by adding the first offset value and the determined output of the inlet pressure sensor. Likewise, in some aspects of the technology, the controller may determine the first pressure value by subtracting the first offset value from the determined output of the inlet pressure sensor. This determined first pressure value will represent a derived external pressure at a point outside of the blood inflow cage of the intracardiac blood pump assembly. Thus, as noted above, where the intracardiac blood pump assembly has been inserted into a patient's right heart such that the blood inflow cage is positioned within the patient's inferior vena cava, and the blood outflow cage is positioned within the patient's pulmonary artery, this determined first pressure value will represent a derived CVP value.

FIG. 6 is a chart showing a set of exemplary inlet pressure offset curves, in accordance with aspects of the disclosure. In that regard, FIG. 6 shows a graph in which the vertical axis represents offset values for the inlet pressure sensor in mm Hg, and the horizontal axis represents output values from the differential pressure sensor in mm Hg. The curves identified as 602, 604, 606, 608, 610, 612, 614, 616, 618, and 620 each correspond to a different motor speed (e.g., the speed of motor 204), and represent, for that motor speed, what offset value (e.g., the first offset value of FIG. 5) should be applied to the reading of the inlet pressure sensor (e.g., the output of inlet pressure sensor 322) for a given reading from the differential pressure sensor (e.g., the output of differential pressure sensor 320).

As noted above, offset curves 602, 604, 606, 608, 610, 612, 614, 616, 618, and 620 may be generated based on any suitable number of data points. For example, in some aspects of the technology, the offset curves may each be generated by connecting two or more points by line segments. In that regard, offset curve 620 may be generated by connecting point 620a to point 620b, and point 620b to point 620c. Likewise, in some aspects of the technology, offset curves may be generated by performing a linear, polynomial, or other suitable regression analysis on a set of data points.

The data points on which offset curves 602, 604, 606, 608, 610, 612, 614, 616, 618, and 620 are based may also be generated in any suitable way. In that regard, in some aspects of the technology, one or more of offset curves 602, 604, 606, 608, 610, 612, 614, 616, 618, and 620 may be based on assumptions regarding how the reading of the inlet pressure sensor will deviate from the actual pressure outside of the blood inflow cage (e.g., blood inflow cage 214) for a given motor speed and differential pressure. For example, offset curves 602, 604, 606, 608, 610, 612, 614, 616, 618, and 620 may be generated based on calculations and/or computer modeling based on the known size, geometry and operational characteristics of the intracardiac blood pump assembly and assumed values for the pumping capacity of the patient's heart, the patient's blood pressure and viscosity, etc.

Likewise, in some aspects of the technology, one or more of offset curves 602, 604, 606, 608, 610, 612, 614, 616, 618, and 620 may be based on data collected from one or more intracardiac blood pump assemblies put in actual use within one or more patients. For example, offset curves 602, 604, 606, 608, 610, 612, 614, 616, 618, and 620 may be based on actual usage data of an intracardiac blood pump assembly equipped with an additional pressure sensor positioned outside the blood inflow cage so that the difference between that additional sensor's reading and the inlet pressure sensor's reading can be directly calculated.

Further, in some aspects of the technology, one more of offset curves 602, 604, 606, 608, 610, 612, 614, 616, 618, and 620 may be based on empirical data collected from a test apparatus designed to model conditions of an intracardiac blood pump assembly operating within a heart. For example, a test apparatus may include a fluid reservoir (e.g., containing human blood, animal blood, or a fluid with similar consistency) connected to a lumen in which the intracardiac blood pump assembly can be inserted. Within that lumen, there may be a valve (e.g., a rubber film or ring) through which the pump is inserted, and an absolute pressure sensor to measure pressure outside of the blood inflow cage of the intracardiac blood pump assembly. A pinch valve or other suitable device may also be installed downstream of the lumen in order to enable outlet pressure to be adjusted to reflect expected operating conditions. Using such a test bed, the intracardiac blood pump assembly can be operated at one or more speeds while data is collected from the inlet pressure sensor (e.g., inlet pressure sensor 322), differential pressure sensor (e.g., differential pressure sensor 320), and the absolute pressure sensor mounted in the lumen. Inlet offset values may be calculated from the difference between the readings of the absolute pressure sensor and the inlet pressure sensor. The calculated inlet offset values may then be correlated to the differential pressure sensor readings, and inlet offset curves (such as those shown in FIG. 6) may be generated based on those data points as discussed above.

FIG. 7 is a flow diagram of an exemplary method 700 for determining an external pressure differential between a point outside of a blood inflow cage and a point outside of a blood outflow cage of an intracardiac blood pump assembly, in accordance with aspects of the disclosure. In that regard, in method 700 it is again assumed that an intracardiac blood pump assembly (e.g., intracardiac blood pump assembly 200 of FIGS. 2-4) has been inserted into a patient's right heart such that the blood inflow cage (e.g., blood inflow cage 214) is positioned within the patient's inferior vena cava, and the blood outflow cage (e.g., blood outflow cage 216) is positioned within the patient's pulmonary artery, and such that the determined external pressure difference will represent the difference between the patient's CVP and PAP. However, here as well, the present technology may likewise be employed in the context of providing left-heart support, in which case the differential pressure sensor (e.g., differential pressure sensor 320) and the inlet pressure sensor (e.g., inlet pressure sensor 322) may be positioned similarly with respect to the blood inflow cage (e.g., blood inflow cage 114), cannula (e.g., cannula 110), and pump housing (e.g., pump housing 107) of an intracardiac blood pump assembly configured for left-heart support (e.g., exemplary intracardiac blood pump assembly 100), and method 700 may be used to instead calculate the external pressure difference between the patient's LVP and the pressure within the patient's aorta (central aortic pressure, or “CAP”).

In step 702, the controller (e.g., controller 302 of FIG. 3) determines the speed at which the motor is operating, and the output of the differential pressure sensor. Here as well, the controller may determine the speed at which the motor is operating in any suitable way, such as from a speed sensor, the voltage being applied to the motor and/or the current being drawn by the motor, a speed setting at which the motor is being operated, etc.

In step 704, the controller determines a second offset value based on the determined speed of the motor, the determined output of the differential pressure sensor, and second data. The second offset value represents the amount by which the reading of the differential pressure sensor is expected to differ from the actual pressure difference between a point outside of the blood inflow cage and a point outside of the blood outflow cage of the intracardiac blood pump assembly, which may be influenced by various factors such as the curvature of the cannula, friction between the blood and the walls of the cannula, dynamic pressure loss within the cannula, turbulence and/or rotational flow caused by the blades of the impeller, etc. Although the second data represents a different offset than the first data, the second data also correlates, for each given motor speed of a plurality of motor speeds, a plurality of differential pressure values to a plurality of offset values, and may be represented in any suitable way. Likewise, any suitable manner of estimating, averaging, or interpolating may be used to determine the second offset value based on the determined speed of the motor, the determined output of the differential pressure sensor, and the second data. Accordingly, the same illustrative and nonlimiting examples described above with respect to step 504 of FIG. 5 may be used for representing the second data, and deriving the second offset value from the second data.

In step 706, the controller determines a second pressure value based on the determined output of the differential pressure sensor and the second offset value. Here as well, the controller may make this determination in any suitable way. For example, in some aspects of the technology, the controller may determine the second pressure value by adding the second offset value and the determined output of the differential pressure sensor. Likewise, in some aspects of the technology, the controller may determine the second pressure value by subtracting the second offset value from the determined output of the differential pressure sensor. This determined second pressure value will represent a derived external pressure difference between a point outside of the blood inflow cage and a point outside of the blood outflow cage of the intracardiac blood pump assembly. Thus, as noted above, where the intracardiac blood pump assembly has been inserted into a patient's right heart such that the blood inflow cage is positioned within the patient's inferior vena cava, and the blood outflow cage is positioned within the patient's pulmonary artery, this determined second pressure value will represent a derived pressure differential between the patient's CVP and PAP.

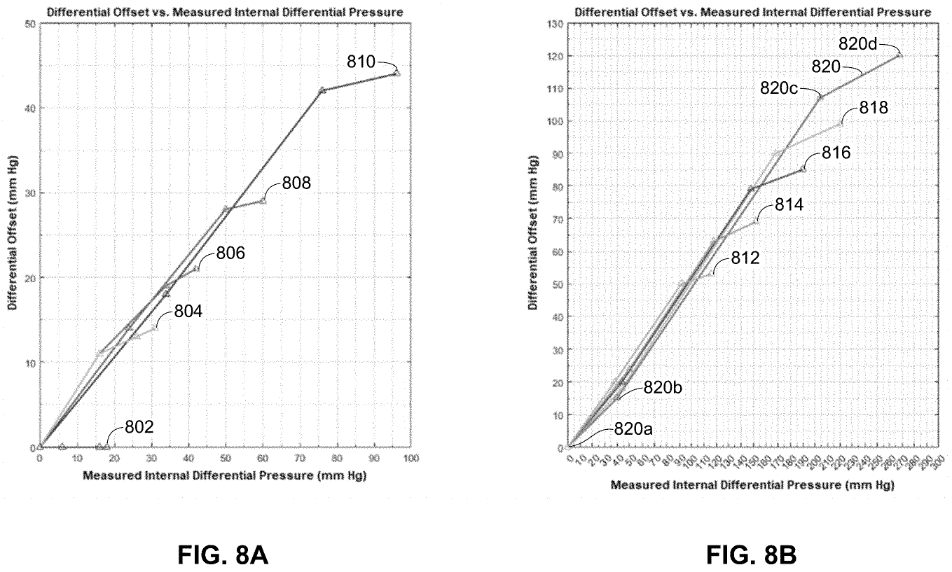

FIGS. 8A and 8B are charts showing a set of exemplary external differential pressure offset curves, in accordance with aspects of the disclosure. In that regard, FIGS. 8A and 8B show two graphs in which the vertical axis represents offset values for the differential pressure sensor in mm Hg, and the horizontal axis represents output values from the differential pressure sensor in mm Hg. The curves identified as 802, 804, 806, 808, 810, 812, 814, 816, 818, and 820 each correspond to a different motor speed (e.g., the speed of motor 204), and represent, for that motor speed, what offset value (e.g., the second offset value of FIG. 7) should be applied to the reading of the differential pressure sensor (e.g., the output of differential pressure sensor 320).

As with the curves of FIG. 6, offset curves 802, 804, 806, 808, 810, 812, 814, 816, 818, and 820 may be generated based on any suitable number of data points. For example, in some aspects of the technology, the offset curves may each be generated by connecting two or more points by line segments. In that regard, offset curve 820 may be generated by connecting point 820a to point 820b, point 820b to point 820c, and point 820c to point 820d. Likewise, in some aspects of the technology, offset curves may be generated by performing a linear, polynomial, or other suitable regression analysis on a set of data points.

Here again, the data points on which offset curves 802, 804, 806, 808, 810, 812, 814, 816, 818, and 820 are based may also be generated in any suitable way. In that regard, in some aspects of the technology, one or more of offset curves 802, 804, 806, 808, 810, 812, 814, 816, 818, and 820 may be based on assumptions regarding how the reading of the differential pressure sensor will deviate from the actual pressure differential between a point outside of the blood inflow cage (e.g., blood inflow cage 214) and a point outside of the blood outflow cage (e.g., blood outflow cage 206) for a given motor speed. For example, offset curves 802, 804, 806, 808, 810, 812, 814, 816, 818, and 820 may be generated based on calculations and/or computer modeling based on the known size, geometry, and operational characteristics of the intracardiac blood pump assembly and assumed values for the pumping capacity of the patient's heart, the patient's blood pressure and viscosity, etc.

Likewise, in some aspects of the technology, one or more of offset curves 802, 804, 806, 808, 810, 812, 814, 816, 818, and 820 may be based on data collected from one or more intracardiac blood pump assemblies put in actual use within one or more patients. For example, offset curves 802, 804, 806, 808, 810, 812, 814, 816, 818, and 820 may be based on actual usage data of an intracardiac blood pump assembly equipped with additional pressure sensors positioned outside the blood inflow cage and blood outflow cage so that a measured external pressure differential can be calculated based on those sensors' readings, and the difference between that measured external pressure differential and the output of the internal differential pressure sensor can be directly calculated.

Further, in some aspects of the technology, one more of offset curves 802, 804, 806, 808, 810, 812, 814, 816, 818, and 820 may be based on empirical data collected from a test apparatus designed to model conditions of an intracardiac blood pump assembly operating within a heart. For example, a similar test apparatus to the one described above may be used. In that regard, the test apparatus may include a fluid reservoir (e.g., containing human blood, animal blood, or a fluid with similar consistency) connected to a lumen in which the intracardiac blood pump assembly can be inserted. Within that lumen, there may be a valve (e.g., a rubber film or ring) through which the pump is inserted, an absolute pressure sensor to measure pressure outside of the blood inflow cage of the intracardiac blood pump assembly, and another absolute sensor to measure pressure outside of the blood outflow cage of the intracardiac blood pump assembly. Here again, a pinch valve or other suitable device may also be installed downstream of the lumen in order to enable outlet pressure to be adjusted to reflect expected operating conditions. Using such a test bed, the intracardiac blood pump assembly can be operated at one or more speeds while data is collected from the differential pressure sensor (e.g., differential pressure sensor 320), as well as the absolute pressure sensors mounted in the lumen. External pressure differential values may then be calculated from the readings of the absolute pressure sensors, and differential offset values may in turn be calculated from the external pressure differential values and the readings of the internal differential pressure sensor. These differential offset values may then be correlated to the differential pressure sensor readings, and external pressure differential offset curves (such as those shown in FIG. 8) may be generated based on those data points as discussed above.

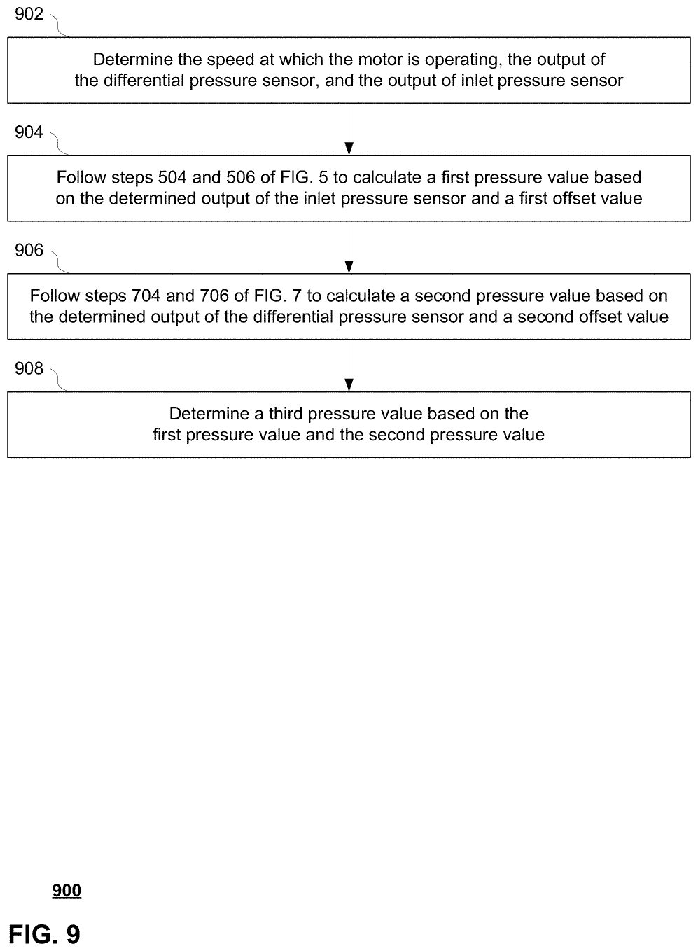

FIG. 9 is a flow diagram of an exemplary method 900 for determining pressure at a point outside of a blood outflow cage of an intracardiac blood pump assembly, in accordance with aspects of the disclosure. In that regard, in method 900 it is again assumed that an intracardiac blood pump assembly (e.g., intracardiac blood pump assembly 200 of FIGS. 2-4) has been inserted into a patient's right heart such that the blood inflow cage (e.g., blood inflow cage 214) is positioned within the patient's inferior vena cava, and the blood outflow cage (e.g., blood outflow cage 216) is positioned within the patient's pulmonary artery, and such that the determined pressure outside of the blood outflow cage will represent the patient's PAP. However, here as well, the present technology may likewise be employed in the context of providing left-heart support, in which case the differential pressure sensor (e.g., differential pressure sensor 320) and the inlet pressure sensor (e.g., inlet pressure sensor 322) may be positioned similarly with respect to the blood inflow cage (e.g., blood inflow cage 114), cannula (e.g., cannula 110), and pump housing (e.g., pump housing 107) of an intracardiac blood pump assembly configured for left-heart support (e.g., exemplary intracardiac blood pump assembly 100), and method 900 may be used to instead calculate the patient's AP.

In step 902, the controller (e.g., controller 302 of FIG. 3) determines the speed at which the motor is operating, the output of the differential pressure sensor, and the output of inlet pressure sensor. Here as well, the controller may determine the speed at which the motor is operating in any suitable way, such as from a speed sensor, the voltage being applied to the motor and/or the current being drawn by the motor, a speed setting at which the motor is being operated, etc.

In step 904, the controller follows steps 504 and 506 of FIG. 5 to calculate a first pressure value based on the determined output of the inlet pressure sensor and a first offset value.

In step 906, the controller follows steps 704 and 706 of FIG. 7 to calculate a second pressure value based on the determined output of the differential pressure sensor and a second offset value.

In step 908, the controller determines a third pressure value based on the first pressure value and the second pressure value. In that regard, in some aspects of the technology, the third pressure value may be calculated by adding the first pressure value and the second pressure value. This determined third pressure value will represent a derived external pressure at a point outside of the blood outflow cage of the intracardiac blood pump assembly. Thus, as noted above, where the intracardiac blood pump assembly has been inserted into a patient's right heart such that the blood inflow cage is positioned within the patient's inferior vena cava, and the blood outflow cage is positioned within the patient's pulmonary artery, this determined third pressure value will represent a derived PAP value.

From the foregoing and with reference to the various figures, those skilled in the art will appreciate that certain modifications can also be made to the present disclosure without departing from the scope of the same. While several aspects of the disclosure have been shown in the figures, it is not intended that the disclosure be limited thereto, as it is intended that the disclosure be as broad in scope as the art will allow and that the specification be read likewise. Therefore, the above description should not be construed as limiting, but merely as exemplifications of particular aspects of the present technology.

1. A intracardiac blood pump system, comprising:
an intracardiac blood pump assembly comprising:
a motor;
a blood inlet;
a blood outlet;
a cannula positioned between the blood inlet and the blood outlet;
a pump housing in fluid communication with the cannula;
an impeller positioned within the pump housing and configured to be driven rotationally by the motor;
a first pressure sensor configured to measure a pressure differential across a wall of the pump housing or the cannula; and
a second pressure sensor positioned within the blood inlet or the pump housing and configured to measure a pressure within the blood inlet or pump housing; and
a controller comprising:
a memory; and
one or more processors coupled to the memory and configured to:
determine a speed of the motor, an output of the first pressure sensor, and an output of the second pressure sensor;
determine a first offset value based on the determined speed of the motor, the determined output of the first pressure sensor, and first data, wherein the first data correlates, for each given motor speed of a plurality of motor speeds, a first plurality of differential pressure values to a first plurality of offset values; and
determine a first pressure value based on the determined output of the second pressure sensor and the first offset value, wherein the first pressure value represents an estimate of a pressure outside the blood inlet.

2. The system of claim 1, wherein the one or more processors are further configured to:
determine a second offset value based on the determined speed of the motor, the determined output of the first pressure sensor, and second data, wherein the second data is different than the first data and correlates, for each given motor speed of a plurality of motor speeds, a second plurality of differential pressure values to a second plurality of offset values; and
determine a second pressure value based on the determined output of the first pressure sensor and the second offset value, wherein the second pressure value represents an estimate of a difference between a pressure outside the blood inlet and a pressure outside the blood outlet.

3. The system of claim 2, wherein the one or more processors are further configured to determine a third pressure value based on the first pressure value and the second pressure value, wherein the third pressure value represents an estimate of the pressure outside the blood outlet.

4. The system of claim 1, wherein the intracardiac blood pump assembly is configured for use in a right heart of a patient.

5. The system of claim 1, wherein the intracardiac blood pump assembly is configured for use in a left heart of a patient.

6. The system of claim 1, wherein the intracardiac blood pump assembly further comprises:
an elongate catheter; and
a motor housing positioned between a distal end of the elongate catheter and a proximal end of the blood inlet, wherein the motor housing is configured to house the motor and to be inserted into a patient.

7. The system of claim 1, wherein the intracardiac blood pump assembly further comprises an elongate catheter, and wherein the motor is configured to drive the impeller via a drive shaft extending through the elongate catheter while the motor remains outside of a patient.

8. A method for operating an intracardiac blood pump system, comprising:
inserting a portion of an intracardiac blood pump assembly into a patient's heart, the intracardiac blood pump assembly comprising:
a motor;
a blood inlet;
a blood outlet;
a cannula positioned between the blood inlet and the blood outlet;
a pump housing in fluid communication with the cannula;
an impeller positioned within the pump housing and configured to be driven rotationally by the motor;
a first pressure sensor configured to measure a pressure differential across a wall of the pump housing or the cannula; and
a second pressure sensor positioned within the blood inlet or the pump housing and configured to measure a pressure within the blood inlet or pump housing;
operating the intracardiac blood pump assembly to pump blood from the blood inlet to the blood outlet;
determining, by one or more processors of a controller, a speed of the motor, an output of the first pressure sensor, and an output of the second pressure sensor;
determining, by the one or more processors, a first offset value based on the determined speed of the motor, the determined output of the first pressure sensor, and first data, wherein the first data correlates, for each given motor speed of a plurality of motor speeds, a first plurality of differential pressure values to a first plurality of offset values; and
determining, by the one or more processors, a first pressure value based on the determined output of the second pressure sensor and the first offset value, wherein the first pressure value represents an estimate of a pressure outside the blood inlet.

9. The method of claim 8, wherein inserting the portion of an intracardiac blood pump assembly into a patient's heart comprises positioning the blood inlet within the patient's inferior vena cava and the blood outlet within the patient's pulmonary artery, and
wherein the first pressure value represents an estimate of the patient's central venous pressure.

10. The method of claim 8, wherein inserting the portion of an intracardiac blood pump assembly into a patient's heart comprises positioning the blood inlet within the patient's left ventricle and the blood outlet within the patient's ascending aorta, and
wherein the first pressure value represents an estimate of the patient's left ventricular pressure.

11. The method of claim 8, further comprising:
determining, by the one or more processors, a second offset value based on the determined speed of the motor, the determined output of the first pressure sensor, and second data, wherein the second data is different than the first data and correlates, for each given motor speed of a plurality of motor speeds, a second plurality of differential pressure values to a second plurality of offset values; and
determining, by the one or more processors, a second pressure value based on the determined output of the first pressure sensor and the second offset value, wherein the second pressure value represents an estimate of a difference between a pressure outside the blood inlet and a pressure outside the blood outlet.

12. The method of claim 11, further comprising determining, by the one or more processors, a third pressure value based on the first pressure value and the second pressure value, wherein the third pressure value represents an estimate of the pressure outside the blood outlet.

13. The method of claim 12, wherein inserting the portion of an intracardiac blood pump assembly into a patient's heart comprises positioning the blood inlet within the patient's inferior vena cava and the blood outlet within the patient's pulmonary artery, and
wherein the third pressure value represents an estimate of the patient's pulmonary artery pressure.

14. The method of claim 12, wherein inserting the portion of an intracardiac blood pump assembly into a patient's heart comprises positioning the blood inlet within the patient's left ventricle and the blood outlet within the patient's ascending aorta, and
wherein the first pressure value represents an estimate of the patient's central aortic pressure.

15. The method of claim 8, wherein the intracardiac blood pump assembly further comprises:
an elongate catheter; and
a motor housing positioned between a distal end of the elongate catheter and a proximal end of the blood inlet, and configured to house the motor; and
wherein inserting a portion of an intracardiac blood pump assembly into a patient's heart comprises inserting the motor housing into the patient.

16. The method of claim 8, wherein the intracardiac blood pump assembly further comprises an elongate catheter, and
wherein operating the intracardiac blood pump assembly to pump blood from the blood inlet to the blood outlet comprises driving the impeller via a drive shaft extending through the elongate catheter while the motor remains outside of a patient.
